(19) United States
(12) Patent Application Publication
(10) Pub. No.: US 2005/0053569 A1
(43) Pub. Date: Mar. 10, 2005

(54) Use of cationic block copolymers to assist the deposition of simple or multiple emulsions

(76) Inventors: Bruno Bavouzet, Gentilly (FR); Matthias Destarac, Paris (FR); Agnieska Wilczewska, Bialystok (FR)

Correspondence Address: Jean Louis Seugnet;Rhodia Inc, Intellectual Property Department, 259 Prospect Plains Road CN 7500, Cranbury, NJ, 08512, US

(21) Appl. No.: 10/498,208

(22) PCT Filed: Dec. 12, 2002
(86) PCT No.: PCT/FR02/04319

(30) Foreign Application Priority Data
Dec. 12, 2001 (FR) 01/16234

(51) Int. Cl. A61K 7/06; A61K 7/11

(52) U.S. Cl. 424/70.17

(57) ABSTRACT

The invention concerns the use to assist in deposition of an emulsion on a surface, of at least a block copolymer comprising at least a non-ionic hydrophobic block and at least a cationic block in conditions of use of said emulsion.

The present invention relates to the use, to assist the deposition of an emulsion on a surface, of at least one block copolymer comprising at least one nonionic hydrophobic block and at least one block containing cationic units, under the conditions of use of said emulsion.

In certain applications, whether in the field of cosmetics, detergency, plant protection or the treatment of metal, it is sought to modify the state of various surfaces by depositing compositions using aqueous formulations, which for the most part arb emulsions.

The present invention relates to this particular field and, more especially, one of the subjects of the invention is the use of certain block copolymers to assist in the deposition of emulsions.

Thus, a first subject of the invention is the use, to assist in the deposition of an emulsion on a surface, of at least one block copolymer comprising at least one nonionic hydrophobic block and at least one block containing cationic units, under the conditions of use of said emulsion; said emulsion comprising an organic phase dispersed in an aqueous phase and optionally comprising at least one outer surfactant and/or at least one outer amphiphilic polymer.

Another subject of the invention consists of a process to assist the deposition of an emulsion on a surface, under the conditions of use of said emulsion, by adding to said emulsion at least one block copolymer comprising at least one nonionic hydrophobic block and at least one block containing cationic units under the conditions of use of said emulsion; said emulsion comprising an organic phase dispersed in an aqueous phase and optionally comprising at least one outer surfactant and/or at least one outer amphiphilic polymer.

Other advantages and characteristics of the present invention will emerge more clearly on reading the description that follows.

Above all, it is indicated that the present invention is suitable for direct simple emulsions, i.e. emulsions comprising an organic phase dispersed in an aqueous phase.

The invention is also suitable for multiple emulsions. According to a first variant, the multiple emulsion comprises an inner aqueous phase dispersed in the organic phase, the combination of inner aqueous phase and organic phase being dispersed in the aqueous phase. According to a second variant, the multiple emulsion comprises an inner organic phase dispersed in the organic phase, the combination of inner organic phase and organic phase being dispersed in the aqueous phase.

In the description hereinbelow, the term “aqueous phase” denotes the aqueous phase of a simple emulsion or the outer aqueous phase of a multiple emulsion; the term “organic phase” denotes the organic phase of a simple emulsion or the organic phase in contact with the outer aqueous phase of a multiple emulsion; the term “inner aqueous phase” denotes the inner aqueous phase of a multiple emulsion; the term “inner organic phase” denotes the inner organic phase of a multiple emulsion.

Moreover, when it is stated that a (co)polymer or a surfactant satisfies the conditions of Bancroft's rule, this means that the fraction of the (co)polymer or of the surfactant that is soluble in the continuous phase of the emulsion is greater than the fraction that is soluble in the dispersed phase of the emulsion; that the two phases of a simple emulsion, or the phases of a multiple emulsion (i.e. the inner phase and the phase in contact with the outer aqueous phase, or the phase in contact with the outer aqueous phase and the outer aqueous phase) are considered in pairs.

More particularly, the (co)polymer or the surfactant is chosen from those that satisfy both of the following conditions simultaneously:
when itis mixed with the aqueous phase, at a concentration of between 0.1% and 10% by weight of said phase at 25° C., it is in the form of a solution in ail or part of the indicated concentration range;
when it is mixed with the organic phase, at a concentration of between 0.1% and 10% by weight of said phase and at 25° C., it is in the form of a dispersion in all or part of the indicated concentration range.

The block copolymer will first be described.

Thus, the block copolymer used in the context of the present invention may be in a linear (multiblock) form, in a branched (comb or grafted) form or in a starburst form.

Linear block copolymers more particularly have a structure comprising two blocks (diblocks).

Copolymers with blocks of branched (comb or grafted) structure preferably have a nonionic skeleton, which is preferably hydrophilic, comprising cationic pendent segments and nonionic hydrophobic segments.

As regards the block copolymers of starburst structure, several possibilities may be envisioned. According to one particular embodiment, if each branch of the starburst is considered, it may comprise either a copolymer containing blocks, preferably a diblock, one of the blocks of which is a nonionic hydrophobic block, the other a cationic block; or a nonionic hydrophobic block or a cationic block.

It is pointed out that each block of the copolymer may consist of a homopolymer or a random or block copolymer or alternatively may have a concentration gradient of the monomers along the chain of the block under consideration.

Moreover, the block copolymer used cannot be considered as a surfactant. Specifically, the weight-average molar mass of the block copolymer is at least 2000 g/mol.

According to one advantageous variant of the invention, the block copolymer used is chosen from those which, when mixed with an aqueous phase, at a concentration of between 0.1% and 10% by weight of said phase at 25° C., are in the form of a solution in all or part of the indicated concentration range.

In addition, the nonionic hydrophobic block(s) of the block copolymer are more particularly obtained from at least one monomer chosen from:
linear, branched, cyclic or aromatic monocarboxylic or polycarboxylic acid esters, comprising at least one ethylenic unsaturation;
saturated carboxylic acid esters comprising 8 to 30 carbon atoms, optionally bearing a hydroxyl group;
α,β-ethylenically unsaturated nitrites, vinyl ethers, vinyl esters, vinylaromatic monomers, vinyl halides or vinylidene halides;
linear or branched, aromatic or non-aromatic hydrocarbon-based monomers comprising at least one ethylenic unsaturation;
propylene oxide or butylene oxide;
alone or as blends, and also the macromonomers derived from such monomers.

It is recalled that the term “macromonomer” denotes a macromolecule bearing one or more functions that may be polymerized via the chosen polymerization method.

It is furthermore pointed out that the hydrophobic blocks of the block copolymer are obtained from monomers free of silicon atoms.

As particular examples of monomers that may be used in the preparation of the hydrophobic block(s) of the block copolymers mention may be made of:
esters of (meth)acrylic acid with an alcohol containing 1 to 12 carbon atoms, for instance methyl (meth)acrylate, ethyl (meth)acrylate, propyl (meth)acrylate, n-butyl (meth)acrylate, t-butyl (meth)acrylate, isobutyl (meth)acrylate, 2-ethylhexyl acrylate or isodecyl acrylate;
vinyl acetate, vinyl Versatate®, vinyl propionate, vinyl chloride, vinylidene chloride, methyl vinyl ether or ethyl vinyl ether;
vinyl nitriles more particularly including those containing from 3 to 12 carbon atoms such as, in particular, acrylonitrile and methacrylonitrile;
sytrene, α-methylstyrene, vinyltoluene, butadiene and chloroprene;
   alone or as blends, and also the macromonomers derived from such monomers.

The preferred monomers are esters of acrylic acid with linear or branched C₁-C₄ alcohols such as methyl (meth)acrylate, ethyl (meth)acrylate, propyl (meth)acrylate and butyl (meth)acrylate, vinyl esters, for instance vinyl acetate, styrene and α-methylstyrene.

It will be added that, according to one advantageous embodiment of the invention, the hydrophobic monomers are chosen such that the polymer corresponding to the hydrophobic block (having a weight-average molar mass similar to that of the block) has a glass transition temperature, Tg, of less than or equal to 50° C.

As regards the block(s) containing cationic units of the block copolymer, these blocks may advantageously be obtained from at least one monomer chosen from:
aminoalkyl (meth)acrylates and aminoalkyl(meth)acrylamides;
monomers comprising at least one secondary, tertiary or quaternary amine function, or a heterocyclic group containing an aromatic or nonaromatic nitrogen atom, vinylamine or ethyleneimine;
diallyldialkylammonium salts;
alone or as blends, or the corresponding salts, and also the macromonomers derived from such monomers.

As more specific examples of cationic monomers from which the block copolymer may be synthesized, mention may be made especially of:
dimethylaminoethyl (meth)acrylate, dimethylaminopropyl (meth)acrylate, di-tert-butylaminoethyl (meth)acrylate, dimethylaminomethyl(meth)acrylamide, dimethylaminopropyl(meth)acrylamide, trimethylammoniumethyl (meth)acrylate chloride, trimethylammoniumethyl acrylate methyl sulfate, benzyldimethylammoniumethyl (meth)acrylate chloride, 4-benzoylbenzyldimethylammoniumethyl acrylate chloride, trimethylammoniumethyl(meth)acrylamide chloride;
ethyleneimine, vinylamine, pyridine, 2-vinylpyridine, 4-vinylpyridine, pyrrolidine, 1-vinyl-2-pyrrolidine, imidazoline, 1-vinyl-3-imidazoline, vinylamine, ethyleneimine;
vinylbenzyltrimethylammonium chloride;
diallyldimethylammonium chloride;
alone or as blends, or the corresponding salts thereof, and also the macromonomers derived from such monomers.

When the monomers are in the form of salts, and more particularly with quaternized amine functions, of ammonium type NR₄⁺ with R, which may be identical or different, representing a hydrogen atom or an alkyl or hydroxyalkyl radical containing 1 to 10 and preferably 1 to 4 carbon atoms, optionally bearing a hydroxyl radical, the counterion may be chosen from halides, for instance chlorine, sulfates, hydrosulfates, alkylsulfates (for example containing 1 to 6 carbon atoms), phosphates, citrates, formates and acetates.

It is pointed out that the cationic block(s) of the block copolymer may be in a non-cationic form in the emulsion, provided that, under the conditions of use of said emulsion, said blocks are in a cationic form.

It is not excluded for either and/or both of the two blocks to comprise one or more nonionic hydrophilic monomers.

Examples of monomers of this type that may be mentioned, inter alia, alone or as a blend, or in the form of macromonomers, include ethylene oxide; linear, branched, cyclic or aromatic monocarboxylic or polycarboxylic acid amides, comprising at least one ethylenic unsaturation or derivatives thereof, for instance (meth)acrylamide or N-methylol(meth)acrylamide; hydrophilic esters derived from (meth)acrylic acid, for instance 2-hydroxyethyl (meth)acrylate; vinyl esters for obtaining polyvinyl alcohol blocks after hydrolysis, for instance vinyl acetate, vinyl Versatate® or vinyl propionate; monomers of the sugar type, for instance osides or highly depolymerized polyholosides.

The term “highly depolymerized” means compounds whose weight-average molecular mass is more particularly less than 20 000 g/mol.

Osides are compounds resulting from the condensation, with elimination of water, of saccharide molecules with each other or of saccharide molecules with non-carbohydrate molecules. Among the osides that are preferred are holosides, which are formed by combining exclusively carbohydrate units and more particularly oligoholosides (or oligosaccharides) comprising only a limited number of these units, i.e. a number generally less than or equal to 10. Examples of oligoholosides that may be mentioned include sucrose, lactose, cellobiose and maltose.

The highly depolymerized polyholosides (ore polysaccharides) that are suitable are described, for example, in the book by P. Arnaud entitled “cours de chimie organique [Course in organic chemistry]”, published by Gauthier-Villars, 1987. Non-limiting examples of highly depolymerized polyholosides that may be mentioned include dextran and starch.

The respective proportions of the various types of monomers, whether they are cationic, nonionic hydrophobic or, where appropriate, nonionic hydrophilic monomers, are such that the resulting block copolymer satisfies Bancroft's rule.

More particularly, the weight-average molar mass of the block copolymer is between 2000 and 100 000 g/mol and preferably between 2000 and 20 000 g/mol. This mass is an absolute mass, determined by steric exclusion chromatography coupled to the MALLS method.

Furthermore, and according to one variant of the invention, the block copolymer is such that the weight-average molar mass of the cationic blocks represents at least twice and preferably at least five times the mass of the nonionic hydrophobic blocks.

In accordance with one preferred embodiment of the invention, the cationic block(s) is (are) such that at least 5 mol % and preferably at least 30 mol % of the monomers from which the block(s) is (are) obtained (number of moles of monomers used in the synthesis of the block under consideration) are cationic monomers. Preferably, the cationic block(s) is (are) obtained from cationic monomers, and even more preferably, the blocks are homopolymers.

Finally, the content of block copolymer in the emulsion represents from 0.1% to 30% by weight relative to the weight of organic phase.

These polymers may be obtained by any known method, whether by controlled or uncontrolled free-radical polymerization, polymerization via ring-opening (especially anionic or cationic polymerization), anionic or cationic polymerization, or alternatively via chemical modification of a polymer.

Living or controlled free-radical polymerization methods are preferably used. As nonlimiting examples of living or controlled polymerization processes, reference may be made especially to patent applications WO 98/58974 (xanthate), WO 97/01478 (dithioesters), WO 99/03894 (nitroxides) and WO 99/31144 (dithiocarbamates).

The grafted or comb block polymers may be obtained via “direct grafting” methods and copolymerization.

Direct grafting consists in polymerizing the selected monomer(s) via a free-radical route, in the presence of the polymer selected to form the skeleton of the final product. If the monomer/skeleton couple and the operating conditions are carefully chosen, then a transfer reaction may take place there between the growing macroradical and the skeleton. This reaction generates a radical on the skeleton, and it is from this radical that the graft grows. The primary radical derived from the initiator may also contribute toward the transfer reactions.

As regards the copolymerization, this involves the grafting in a first stage, onto the end of the future pendent segment, of a free-radical-polymerizable function. This grafting may be performed via common methods of organic chemistry. Next, in a second stage, the macromonomer thus obtained is polymerized with the monomer chosen to form the skeleton and a “comb” polymer is obtained.

In the case of polymers of starburst type, the syntheses may be essentially classified into two groups. The first corresponds to the formation of the arms of the polymers from a multifunctional compound constituting the center (“core-first” technique) (Kennedy, J. P. et al., Macromolecules, 29, 8631 (1996), Deffieux, A. et al., Ibid, 25, 6744 (1992), Gnanou, Y. et al., Ibid, 31, 6748 (1998)) and the second corresponds to a method in which the polymer molecules which will constitute the arms are first synthesized and then linked together on a core to form a star-shaped polymer (“arm-first” technique). Among the methods that may be used for the arms, mention may be made especially of the method involving the reaction of these arms with a compound containing a plurality of functional groups capable of reacting with antagonistic functional end groups of said arms (Fetters, L. J. et al., Macromolecules, 19, 215 (1986), Hadjichristidis, N. et al., Macromolecules, 26, 2479 (1993), Roovers, J. et al., Macromolecules, 26, 4324 (1993)). Mention is also made of the method involving the addition of a compound containing a plurality of polymerizable groups, followed by polymerization of said arms (Rempp, P. et al., Polym. Sci. Part C, 22, 145 (1968), Fetters, L. J. et al., Macromolecules, 8, 90 (1975), Higashimura et al., Ibid, 24, 2309 (1991)).

To obtain polymer chains subsequently constituting the arms of the starbursts, use is generally made of methods for controlling the polymerization reaction. Thus, living anionic and cationic polymerizations are the methods that are currently the most commonly used.

In accordance with a first embodiment of the invention, the block copolymer that has just been described is used to assist the deposition of a simple emulsion comprising an organic phase dispersed in an aqueous phase.

The compound used as organic phase is preferably chosen from compounds whose solubility in water does not exceed 10% by weight, over a temperature range of between 20° C. and the temperature of preparation of the emulsion.

Suitable compounds that may be mentioned include organic or organosilicon compounds, especially organic oils of animal or plant origin, or mineral oils, and also waxes derived from the same origins, or mixtures thereof, and linear, cyclic, branched or crosslinked polyorganosiloxane resins, waxes or oils.

As organic oils of animal origin, mention may be made, inter alia, of sperm whale oil, whale oil, seal oil, sardine oil, herring oil, shark oil and cod liver oil; pig or sheep fat (tallow).

As waxes of animal origin, mention may be made of beeswax.

As examples of organic oils of plant origin, mention may be made, inter alia, of rapeseed oil, sunflower oil, groundnut oil, olive oil, walnut oil, maize oil, soybean oil, linseed oil, hemp oil, grapeseed oil, coconut oil, palm oil, cottonseed oil, babassu oil, jojoba oil, sesame seed oil, castor oil, cocoa butter and shea butter.

As waxes of plant origin, mention may be made of carnauba wax.

As regards the mineral oils, mention may be made, inter alia, of petroleum fractions, naphthenic oils and liquid paraffins (petroleum jelly). Paraffin waxes may also be suitable for preparing the emulsion.

The products derived from the alcoholysis of the abovementioned oils may also be used.

It would not constitute a departure from the context of the present invention to use, as organic phase, at least one saturated or unsaturated fatty acid, at least one saturated or unsaturated fatty alcohol, at least one fatty acid ester, or mixtures thereof.

More particularly, said acids contain 8 to 40 carbon atoms, more particularly 10 to 40 carbon atoms and preferably 18 to 40 carbon atoms, and may comprise one or more conjugated or nonconjugated ethylenic unsaturations, and optionally one or more hydroxyl groups. As regards the alcohols, they may comprise one or more hydroxyl groups.

Examples of saturated fatty acids that may be mentioned include palmitic acid, stearic acid and behenic acid.

Examples of unsaturated fatty acids that may be mentioned include myristoleic acid, palmitoleic acid, oleic acid, erucic acid, linoleic acid, linolenic acid, arachidonic acid and ricinoleic acid, and also mixtures thereof.

As regards the alcohols, they more particularly contain 4 to 40 and preferably 10 to 40 carbon atoms, optionally one or more conjugated or nonconjugated ethylenic unsaturations and optionally several hydroxyl groups. Polymers comprising several hydroxyl groups may also be suitable, for instance polypropylene glycols.

Examples of alcohols that may be mentioned include those corresponding to the abovementioned acids.

As regards the fatty acid esters, these may advantageously be obtained from fatty acids chosen from the compounds named above. The alcohols from which these esters are prepared more particularly contain 1 to 6 carbon atoms. Preferably, they are methyl, ethyl, propyl or isopropyl esters.

Moreover, it is not excluded to use monoglycerides, diglycerides and triglycerides as organic phase.

They may also be chosen from silicone oils, advantageously totally or partially consisting of units of formula
R′₃₋ₐRₐSiO_{1/2} (unit M) and/or R₂SiO (unit D)
in which formulae:
a is an integer from 0 to 3;
the radicals R are identical or different and represent:
   a saturated or unsaturated aliphatic hydrocarbon-based group containing from 1 to 10 carbon atoms;
   an aromatic hydrocarbon-based group containing from 6 to 13 carbon atoms;
   a polar organic group linked to silicon via an Si—C or Si—O—C bond;
the radicals R′ are identical or different and represent:
   a saturated or unsaturated aliphatic hydrocarbon-based group containing from 1 to 10 carbon atoms;
   an aromatic hydrocarbon-based group containing from 6 to 13 carbon atoms;
   an —OH function;
   an amino-functional or amido-functional group containing from 1 to 6 carbon atoms, linked to silicon via an Si—N bond.

Preferably, at least 80% of the radicals R represent a methyl group.

These silicones may optionally comprise preferably less than 5 mol % of units of formulae T and/or Q:
RSiO_{3/2} (unit T) and/or SiO₂ (unit Q)
in which formula R has the definition given above.

Examples of aliphatic or aromatic hydrocarbon-based radicals R that may be mentioned include the following groups:
alkyl, preferably optionally halogenated C₁-C₁₀ alkyl, such as methyl, ethyl, octyl or trifluoropropyl;
alkoxyalkylene, more particularly of C₂-C₁₀ and preferably of C₂-C₅, such as —CH₂—CH₂—O—CH₃;
alkenyls, preferably C₂-C₁₀ alkenyl, such as vinyl, allyl, hexenyl, decenyl or decadienyl;
alkenyloxyalkylene such as —(CH₂)₃—O—CH₂—CH═CH₂, or alkenyloxyalkoxyalkyl such as —(CH₂)₃—OCH₂—CH₂—O—CH═CH₂ in which the alkyl portions are preferably of C₁-C₁₀ and the alkenyl portions are preferably of C₂-C₁₀;
aryls, preferably of C₆-C₁₃, such as phenyl.

Examples of polar organic groups R that may be mentioned include:
hydroxy-functional groups such as alkyl groups substituted with one or more hydroxyl or di(hydroxyalkyl)amino groups and optionally interrupted with one or more divalent hydroxyalkylamino groups. The term “alkyl” means a hydrocarbon-based chain preferably of C₁-C₁₀ and better still of C₁-C₆; examples of these groups are —(CH₂)₃—OH; —(CH₂)₄N—(CH₂CH₂OH)₂; —(CH₂)₃—N(CH₂CH₂OH)—CH₂—CH₂—N(CH₂CH₂OH)₂;
amino-functional groups such as alkyl substituted with one or more amino or amino-alkylamino groups in which alkyl is as defined above; examples of these are —(CH₂)₃—NH₂; (CH₂)₃—NH—(CH₂)₂NH₂;
amido-functional groups such as alkyl substituted with one or more acylamino groups and optionally interrupted with one or more divalent alkyl-CO—N< groups in which alkyl is as defined above and acyl represents alkylcarbonyl; an example is the —(CH₂)₃—N(COCH₃)—(CH₂)₂NH(COCH₃) group;
carboxy-functional groups, such as carboxyalkyl optionally interrupted with one or more oxygen or sulfur atoms, in which alkyl is as defined above; an example is the —CH₂—CH₂—S—CH₂—COOH group.

Examples of radicals R′ that may be mentioned include:
alkyl groups, preferably optionally halogenated C₁-C₁₀ alkyl such as methyl, ethyl, octyl or trifluoropropyl;
aryl groups, preferably of C₆-C₁₃, such as phenyl;
amino-functional groups such as alkyl or aryl, substituted with amino, alkyl preferably being of C₁-C₆ and aryl denoting a cyclic aromatic hydrocarbon-based group preferably of C₆-C₁₃ such as phenyl; examples of these are ethylamino and phenylamino;
amido-functional groups such as alkylcarbonylamino in which alkyl is preferably of C₁-C₆; an example of these is methylacetamido.

Concrete examples of “units D” that may be mentioned include: (CH₃)₂SiO; CH₃(CH═CH₂)SiO; CH₃(C₆H₅)SiO; (C₆H₅)₂SiO; CH₃(CH₂—CH₂—CH₂OH)SiO.,

Concrete examples of “units M” that may be mentioned include: (CH₃)₃SiO_{1/2}; (CH₃)₂(OH)SiO_{1/2}; (CH₃)₂ (CH═CH₂)SiO_{1/2}; (OCH₃)₃SiO_{1/2}; [O—C(CH₃)═CH₂]₃SiO_{1/2}; [ON═C(CH₃)]₃SiO_{1/2}; (NH—CH₃)₃SiO_{1/2}; (NH—CO—CH₃)₃SiO_{1/2}.

Concrete examples of “units T” that may be mentioned include: CH₃SiO_{3/2}; (CH═CH₂)SiO_{3/2}.

When the silicones contain reactive and/or polar radicals R (such as OH, vinyl, allyl, hexenyl, aminoalkyl, etc.), these radicals generally represent not more than 5% of the weight of the silicone and preferably not more than 1% of the weight of the silicone.

Volatile oils, for instance hexamethyldisiloxane, octamethyldisiloxane, decamethyltetrasiloxane, dodecamethylpentasiloxane, tetradecamethylhexasiloxane, hexadecamethylhexasiloxane; heptamethyl-3[(trimethylsilyl)oxy]trisiloxane, hexamethyl-3,3-bis-[(trimethylsilyl)oxy]trisiloxane; hexamethylcyclotrisiloxane, octamethylcyclotetrasiloxane, decamethylcyclopentasiloxane, dodecamethylcyclohexasiloxane, pentamethyl[(trimethylsilyl)oxy]cyclotrisiloxane, may preferably be used.

Similarly, nonvolatile silicones may be used, for instance polydimethylsiloxane and α,ω-bis(hydroxy)polydimethylsiloxane oils and gums and also polydimethylsiloxane, polyphenylmethylsiloxane and α,ω-bis(hydroxy)polydimethylsiloxane gums may be used.

α,ω-bis(trimethyl)polydimethylsiloxane oils and α,ω-bis(hydroxy)polydimethylsiloxane oils are more particularly preferred.

As representative silicones that are most particularly suitable for the present invention, mention may be made especially of silicones of polydimethylsiloxane (dimethicone) and diphenyl dimethicone type.

Another type of aminosilicones that are particularly suitable for the present invention are silicones containing sterically hindered piperidyl groups. The sterically hindered piperidyl groups may be represented by formulae I and II below:
the sterically hindered piperidyl groups of formula I 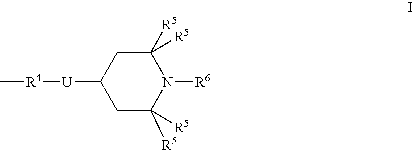
   in which
R⁴ is a divalent hydrocarbon-based radical chosen from:
   linear or branched alkylene radicals containing 2 to 18 carbon atoms;
   alkylenecarbonyl radicals in which the linear or branched alkylene portion contains 2 to 20 carbon atoms;
   alkylene-cyclohexylene radicals in which the linear or branched alkylene portion contains 2 to 12 carbon atoms and the cyclohexylene portion comprises an OH group and optionally 1 or 2 alkyl radicals containing 1 to 4 carbon atoms;
   radicals of formula —R⁷O—R⁷ in which the radicals R⁷, which may be identical or different, represent alkylene radicals containing from 1 to 12 carbon atoms;
   the radicals of formula —R⁷—O—R⁷ in; which the radicals R⁷ have the meanings given above and either or both of them is (are) substituted with one or two —OH group(s);
   the radicals of formula —R⁷—COO—R⁷ in which the radicals R⁷ have the meanings given above;
   the radicals of formula —R⁸—O—R⁹—O—CO—R⁸ in which the radicals R⁸ and R⁹, which may be identical or different, represent alkylene radicals containing 2 to 12 carbon atoms and the radical R⁹ is optionally substituted with a hydroxyl radical;
   U represents —O— or —NR¹⁰—, R¹⁰ being a radical chosen from a hydrogen atom, a linear or branched alkyl radical containing 1 to 6 carbon atoms and a divalent radical of formula: 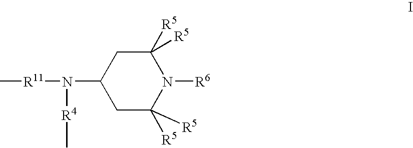
   in which R⁴ has the meaning given above, R⁵ and R⁶ have the meanings given below and R¹¹ represents a linear or branched divalent alkylene radical containing from 1 to 12 carbon atoms, one of the valency bonds (that of R¹¹) being linked to the atom of —NR¹⁰—, the other (that of R⁴) being linked to a silicon atom;
the radicals R⁵ are identical or different and are chosen from linear or branched alkyl radicals containing 1 to 3 carbon atoms and a phenyl radical;
the radical R⁶ represents a hydrogen radical or the radical R⁵ or O.;
or the sterically hindered piperidyl groups of formula II 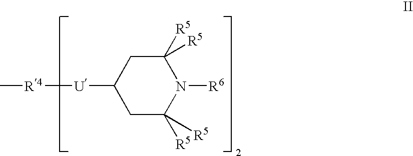
   in which
R′⁴ is chosen from a trivalent radical of formula: 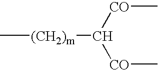
in which m represents a number from 2 to 20, and a trivalent radical of formula: 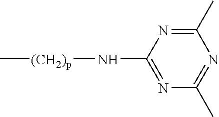
in which p represents a number from 2 to 20;
U′ represents —O— or NR¹²—, R¹² being a radical chosen from a hydrogen atom and a linear or branched alkyl radical containing 1 to 6 carbon atoms;
   R⁵ and R⁶ have the same meanings as those given above for formula I.

Said polyorganosiloxanes containing a sterically hindered piperidyl function are, preferably especially those that may be prepared according, to the process described in EP-A-659 930.

Most preferably said polyorganosiloxane containing a sterically hindered piperidyl function is a linear, cyclic or three-dimensional polyorganosiloxane of formula: 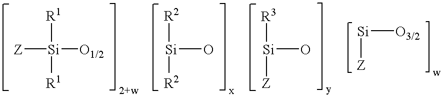
in which:
(1) the symbols Z, which may be identical or different, represent R¹ below and/or B;
(2) the symbols R¹, R² and R³, which may be identical or different, represent a monovalent hydrocarbon-based radical chosen from linear or branched alkyl radicals containing from 1 to 4 carbon atoms, linear or branched alkoxy radicals containing from 1 to 4 carbon atoms, a phenyl radical and, preferably, a hydroxyl radical, an ethoxy radical, a methoxy radical or a methyl radical;
(3) the symbols B, functional groups which may be identical and/or different, represent a group containing sterically hindered piperidyl function(s) chosen from those mentioned above; and
(4) the number of units ηSi without a group B ranges from 10 to 450, preferably from 50 to 250;
   the number of units ηSi with a group B ranges from 1 to 5 and preferably from 1 to 3;
   0≦w≦10 and 8<y<448.

Most preferably, said polyorganosiloxane containing sterically hindered piperidyl function(s) is linear.

Finally, the organic phase may comprise an amount of water that does not exceed the water solubility limit in said organic phase (at a temperature of between 20° C. and the emulsion preparation temperature).

The organic phase may also comprise at least one active material.

Said active materials are in liquid form, soluble in the organic phase, dissolved in anorganic solvent that is miscible in the organic phase, or alternatively in the form of a solid dispersed in said phase.

More particularly, the active materials are such that their solubility in water does not exceed 10% by weight between 20° C. and the emulsion preparation temperature.

In addition, the active materials preferably have a melting point of less than or equal to 100° C. and more particularly less than or equal to 80° C.

Examples of active materials in the cosmetics sector that may be mentioned include silicone oils belonging, for example, to the dimethicone family; lipophilic vitamins, for instance vitamin A.

In the paper sector, examples that may be mentioned include size resins and water-repellent resins, such as alkylketene dimer (AKD) or alkenyl-succinic anhydride (ASA).

In the detergency sector, possible active materials that may be mentioned include silicone antifoams, mineral and plant oils, and aminiosilicones.

In the agrochemistry sector, the plant-protection active materials may be chosen from the family of α-cyanophenolxybenzyl carboxylates or α-cyanohalophenoxycarboxylates, the family of N-methylcarbonates comprising aromatic substituents, and active materials such as aldrin, azinphos-methyl, benfluralin, bifenthrin, chlorphoxim, chlorpyrifos, fluchloralin, fluroxypyr, dichlorvos, malathion, molinate, parathion, permethrin, profenofos, propiconazole, prothiofos, pyrifenox, butachlor, metolachlor, chlorimephos, diazinon, fluazifop-P-butyl, heptopargil, mecarbam, propargite, prosulfocarb, bromophos-ethyl, carbophenothion or cyhalothrin.

It is similarly possible to use active materials such as those included in the composition of metal-working or metal-bending lubricants. The active material is usually an oil, an oil derivative or a fatty acid ester.

The active material may also be chosen from organic solvents or mixtures of such solvents that are sparingly miscible or immiscible in water, especially such as those used for cleaning or stripping, such as aromatic petroleum fractions, terpene compounds such as D- or L-limonenes, and solvents such as Solvesso®. Hydrocarbon-based oils, for instance liquid petroleum jelly, chlorinated solvents and C₁-C₄ alkyl diesters of at least one C₄-C₆ aliphatic diacid are also suitable as solvents. Mixtures of diacid esters, which are esters derived essentially from adipic acid, glutaric acid and succinic acid, the alkyl groups of the ester portion being chosen especially from methyl and ethyl groups, but also possibly being propyl, isopropyl, butyl, n-butyl and isobutyl; anisole; n-methylpyrrolidone; dimethyl sulfoxide; ketones, for instance cyclopentanone or methyl isobutyl ketone; polyalkylene glycols, for instance polyethylene glycol 400 or polypropylene, glycol 400, are more particularly used.

In the case where the organic phase comprises one or more hydrophobic active materials that are different from the organic phase, their content more particularly represents 10% to 50% by weight of said inner organic phase.

Finally, the organic phase itself, as described previously, may be considered as a hydrophobic active material.

In addition, the outer aqueous phase may comprise at least one outer surfactant and/or at least one outer amphiphilic polymer.

More particularly, the surfactants and polymers satisfy Bancroft's rule mentioned previously.

According to a first variant, the outer surfactant and/or outer amphiphilic polymer are chosen from nonionic surfactants and nonionic amphiphilic polymers, optionally combined with at least one anionic surfactant and/or at least one anionic amphiphilic polymer.

According to this first variant, the total content of outer surfactant(s) and/or outer amphiphilic polymer(s) is between 0.5% and 10% by weight and preferably between 1% and 5% by weight relative to the inverse emulsion; the amount of anionic surfactant and/or anionic amphiphilic polymer, if they are present, represents 0.5% to 5% by weight and preferably 0.5% to 2% by weight relative to the weight of nonionic surfactant and/or nonionic amphiphilic polymer.

As regards the polyalkoxylated nonionic surfactants, compounds for which the number of alkoxylated, more particularly ethoxylated and/or propoxylated units is such that the HLB value is greater than or equal to 10 are used.

By way of illustration, the following surfactants, alone or as a mixture, are suitable:
alkoxylated fatty alcohols
alkoxylated triglycerides
alkoxylated fatty acids
alkoxylated sorbitan esters
alkoxylated fatty amines
alkoxylated bis(1-phenylethyl)phenols
alkoxylated tris(1-phenylethyl)phenols
alkoxylated alkylphenols.

The alkoxylated fatty alcohols generally contain from 6 to 22 carbon atoms, the alkoxylated units being excluded from these numbers.

The alkoxylated triglycerides may be triglycerides of plant or animal origin.

The alkoxylated sorbitan esters are cyclized sorbitol esters of a fatty acid containing from 10 to 20 carbon atoms, for instance lauric acid, stearic acid or oleic acid.

The alkoxylated fatty amines generally contain from 10 to 22 carbon atoms, the alkoxylated units being excluded from these numbers.

The alkoxylated alkylphenols generally contain one or two linear or branched alkyl groups containing 4 to 12 carbon atoms. Examples that may especially be mentioned include octyl, nonyl or dodecyl groups.

As regards the nonionic amphiphilic polymers, polyalkoxylated compounds comprising at least two blocks, one of them being hydrophilic and the other hydrophobic; at least one of the blocks comprising polyalkoxylated units, more particularly polyethoxylated and/or polypropoxylated units, are preferably used.

That which has been stated previously in the context of the description of the nonionic hydrophilic monomers and of the hydrophobic monomers that may be used for the preparation of the block copolymers included in the composition of the emulsion remains valid and will not be repeated here.

Examples of polymers of this type that may be mentioned, inter alia, include polyethylene glycol/polypropylene glycol/polyethylene glycol triblock polymers. Such polymers are well known and are especially sold under the brand names Pluronic (sold by BASF) and Arlatone (sold by ICI).

According to another embodiment, the nonionic amphiphilic polymer is an amphiphilic block polymer obtained by polymerization of at least one nonionic hydrophilic monomer and of at least one hydrophobic monomer, the proportion and nature of said monomers being such that the resulting polymer satisfies the conditions of Bancroft's rule.

These amphiphilic polymers furthermore comprise at least one hydrophobic block and at least one nonionic hydrophilic block.

In the case where said polymer comprises at least three blocks, and more particularly three blocks, the polymer is advantageously linear. In addition, the hydrophilic blocks are more particularly located at the ends.

In the case where the polymers comprise more than three blocks, these blocks are preferably in the form of grafted or comb polymers.

The lists of nonionic hydrophilic monomers and of nonionic hydrophobic monomers, and also the various preparation methods, cited in the context of the description of the amphiphilic block polymers, may be repeated in the case of the polymers according to this variant.

However, the preferred hydrophilic monomers are acrylamide and methacrylamide, alone or as a mixture, or in the form of macromonomers; the preferred monomers are acrylic acid esters with linear or branched C₁-C₄ alcohols, such as methylacrylate, ethyl acrylate, propyl acrylate and butyl acrylate, and vinyl esters, for instance vinyl acetate, styrene and α-methylstyrene.

The nonionic surfactant and/or nonionic amphiphilic polymer that have rust been described may be combined with at least one anionic surfactant and/or anionic amphiphilic polymer, among which the following may be used, alone or as mixtures:
alkyl ester sulfonates, for example of formula R—CH(SO₃M)-COOR, in which R represents a C₈-C₂₀ and preferably C₁₀-C₁₆ alkyl radical, R′ represents a C₁-C₆ and preferably C₁-C₃ alkyl radical and M represents an alkali metal cation (sodium, potassium or lithium), substituted or unsubstituted ammonium (methyl-, dimethyl-, trimethyl- or tetramethylammonium, dimethylpiperidinium, etc.) or an alkanolamine derivative (monoethanolamine, diethanolamine, triethanolamine, etc.). Mention may be made most particularly of methyl ester sulfonates in which the radical R is of C₁₄-C₁₆; alkyl benzenesulfonates, more particularly of C₉-C₂₀, primary or secondary alkyl sulfonates, especially of C₈-C₂₂ alkylglyceryl sulfonates;
alkyl sulfates, for example of formula ROSO₃M, in which R represents a C₁₀-C₂₄ and preferably C₁₂-C₂₀ alkyl or hydroxyalkyl radical; M representing a hydrogen atom or a cation of the same definition as above, and also the polyalkoxylated (ethoxylated and/or propoxylated) derivatives thereof, for instance sodium dodecyl sulfate;
alkyl ether sulfates, for example of formula RO(CH₂CH₂O)ₙSO₃M in which R represents a C₁₀-C₂₄ and preferably C₁₂-C₂₀ alkyl or hydroxyalkyl radical; M representing a hydrogen atom or a cation of the same definition as above, n generally ranging from 1 to 4, and also the polyalkoxylated (ethoxylated and/or propoxylated) derivatives thereof, for instance lauryl ether sulfate with n=2;
alkylamide sulfates, for example of formula RCONHROSO₃M in which R represents a C₂-C₂₂ and preferably C₆-C₂₀ alkyl radical, R′ represents a C₂-C₃ alkyl radical, M representing a hydrogen atom or a cation of the same definition as above, and also the polyalkoxylated (ethoxylated and/or propoxylated) derivatives thereof;
saturated or unsaturated fatty acid salts, for instance those of C₈-C₂₄ and preferably of C₁₄-C₂₀, N-acyl N-alkyltaurates, alkyl isethionates, alkyl succinamates and alkyl sulfosuccinates, sulfosuccinate monoesters or diesters, N-acyl sarcosinates and polyethoxycarboxylates; and
alkyl and/or alkyl ether and/or alkylaryl ether ester phosphates.

It is pointed out that in the case where an anionic species (surfactant) is placed in contact with the block copolymer, whether during the preparation of the emulsion, after this emulsion has been manufactured or during the preparation of the formulation into which the emulsion is introduced, the block copolymer and the anionic species are of such a nature and are used in a concentration such that they are compatible. The term “compatible” means that there is no macroscopic phase separation at 20° C. after one hour.

Among the anionic polymers that may be used, mention may be made most particularly of block, preferably diblock or triblock, polymers obtained by polymerization of at least one anionic hydrophilic monomer, optionally of at least one nonionic hydrophilic monomer, and of at least one hydrophobic monomer.

In this case also, the choice of monomers and the respective proportions thereof are such that the resulting polymer satisfies the conditions of Bancroft's rule.

The nonionic and anionic hydrophilic monomers, the hydrophobic monomers and the synthesis methods cited in the context of the description of the inner amphiphilic polymers may be used to obtain the outer amphiphilic polymers according to this variant. Reference may thus be made thereto.

A second variant of the invention consists in using as outer surfactant and/or outer amphiphilic polymer one or more anionic amphiphilic polymers optionally combined with at least one anionic surfactant; the total content of anionic amphiphilic polymer and/or anionic surfactant is between 0.5% and 10% by weight and preferably between 1% and 5% by weight relative to the inverse emulsion.

Reference may be made to the lists indicated for the first variant of outer surfactants and/or outer polymers as regards the surfactants and/or polymers in accordance with this variant.

Moreover, the organic phase/aqueous phase weight ratio is more particularly between 0.1/99.9 and 90/10.

In accordance with a second embodiment of the invention, the block copolymer that has just been described is used to assist the deposition of a multiple emulsion.

According to this embodiment of the invention, the subject of a first variant is multiple emulsions comprising an inner aqueous phase dispersed in the organic phase, the inverse emulsion (combination of the inner aqueous phase and of the organic phase) being dispersed in the aqueous phase. Furthermore, the inner aqueous phase and the organic phase comprise at least one inner surfactant and/or at least one inner amphiphilic polymer.

The inner aqueous phase may optionally comprise at least one hydrophilic active material.

This hydrophilic active material may be in a liquid form; in a form dissolved in a water-miscible solvent, for instance ethanol, propylene glycol or glycerol; or in a solid form.

The content of hydrophilic active material is more particularly between 0.1% and 50% by weight of the inner aqueous phase and preferably between 0.1% and 20% by weight of the inner aqueous phase.

As examples of active materials that may be used in the cosmetics sector, mention may be made of substances that have a cosmetic effect, a therapeutic effect or any other substance that may be used for treating the skin and the hair.

Thus, active materials that may be used include skin and hair conditioners, especially such as polymers comprising quaternary ammoniums which may optionally be engaged in heterocycles (compounds of the quaternium or polyquaternium type, etc.), humectants; fixing (styling) agents more particularly chosen from polymers (homopolymers, copolymers or terpolymers, for instance acrylamide, acrylamide/sodium acrylate, polystyrene sulfonate, etc.), cationic polymers, polyvinylpyrrolidone, polyvinyl acetate, etc.

It is similarly possible to use dyes; astringents, which may be used in deodorants and which are more particularly aluminum or zirconium salts; antibacterial agents; antiinflammatory agents; anesthetics; sunscreens, etc.

Mention may also be made of α- and β-hydroxy acids, for instance citric acid, lactic acid, glycolic acid and salicylic acid; dicarboxylic acids, which are preferably unsaturated and containing 9 to 16 carbon atoms, for instance azelaic acid; vitamin C and its derivatives, especially glycosyl and phosphate derivatives; biocides, especially cationic biocides (Glokill PQ and Rhodoquat RP50, sold by Rhodia Chimie), as active materials that are suitable in cosmetic formulations.

As active materials that are suitable in the papermaking sector, mention may be made especially of calcium chloride and hydrochloric acid.

In accordance with one particularly advantageous embodiment, the inner aqueous phase may comprise at least one additive chosen from salts such as alkali metal or alkaline-earth metal halides (for instance sodium chloride or calcium chloride), or alkali metal or alkaline-earth metal sulfates (for instance sodium sulfate), or mixtures thereof. As possible additives of the inner aqueous phase, mention may also be made of sugars, for instance glucose, or polysaccharides, especially such as dextran, or mixtures thereof.

The concentration of additive of the salt type, when this additive is present, is more particularly between 0.05 and 1 mol/l and preferably 0.1 to 0.4 mol/l.

The concentration of additive of the sugar and/or polysaccharide type is such that the osmotic pressure of the inner aqueous phase comprising the sugar and/or polysaccharide corresponds to the osmotic pressure of an inner aqueous phase comprising 0.05 to 1 mol/l of salt.

As regards the organic phase, and more particularly its nature and also that of the active material(s) it may comprise, reference may be made to what has been described in this respect in the part of the description relating to simple emulsions.

As indicated previously, the organic phase and the inner aqueous phase comprise at least one inner surfactant and/or inner amphiphilic polymer.

According to a first possibility, said surfactant and/or polymer satisfy Bancroft's rule. In other words, the fraction of the surfactant or polymer that is soluble in the continuous phase of the inverse emulsion is greater than the fraction that is soluble in the dispersed phase of the inverse emulsion.

More particularly, the amphiphilic surfactant and/or amphiphilic polymer are chosen from nonionic surfactants and/or amphiphilic block polymers.

As examples of nonionic surfactants that may be included in the composition of the inverse emulsion, mention may be made, alone or as a mixture, of surfactants chosen from:
alkoxylated fatty alcohols
alkoxylated monoglycerides, diglycerides and triglycerides
alkoxylated fatty acids
optionally alkoxylated sorbitan esters
alkoxylated fatty amines
alkoxylated bis(1-phenylethyl)phenols
alkoxylated tris(1-phenylethyl)phenols
alkoxylated alkylphenols
   the number of alkoxylated (ethoxylated, propoxylated or butoxylated) units is such that the HLB value is less than or equal to 8.

As regards the amphiphilic block polymer, this polymer comprises at least two blocks, at least one of which is a hydrophobic block and at least one of which is a neutral, anionic or cationic hydrophilic block.

In the case where the amphiphilic polymer comprises at least three blocks, and more particularly three blocks, the polymer is preferably linear. In addition, the hydrophobic blocks are more particularly located at the ends.

In the case where the polymers comprise more than three blocks, these blocks are preferably in the form of grafted or comb polymers.

These inner amphiphilic polymers may be synthesized by using the polymerization methods mentioned in the context of the description of the block copolymer, and reference may moreover be made to the various lists of nonionic hydrophobic monomers, nonionic hydrophilic monomers and cationic monomers detailed on this occasion.

Nonionic hydrophobic monomers that may especially be mentioned include:
linear, branched, cyclic or aromatic monocarboxylic or polycarboxylic acid esters, comprising at least one ethylenic unsaturation,
saturated carboxylic acid esters containing 8 to 30 carbon atoms, optionally bearing a hydroxyl group,
α,β-ethylenically unsaturated nitriles, vinyl ethers, vinyl esters, vinylaromatic monomers, vinyl halides or vinylidene halides,
linear or branched, aromatic or nonaromatic hydrocarbon-based monomers comprising at least one ethylenic unsaturation,
monomers of cyclic or noncyclic siloxane type, and chlorosilanes;
propylene oxide or butylene oxide;
   alone or as mixtures, and also macromonomers derived from such monomers.

As regards the nonionic hydrophilic monomers, the ones that are especially suitable are ethylene oxide; linear, branched cyclic or aromatic monocarboxylic or polycarboxylic acid amides comprising at least one ethylenic unsaturation, or derivatives thereof, for instance (meth)acrylamide, N-methylol(meth)acrylamide; hydrophilic esters derived from (meth)acrylic acid for instance 2-hydroxyethyl (meth)acrylate; vinyl esters for obtaining polyvinyl alcohol blocks after hydrolysis, for instance vinyl acetate, vinyl Versatate® or vinyl propionate; monomers of the sugar type, for instance osides and highly depolymerized polyholosides.

As regards the monomers, they may be chosen advantageously from:
aminoalkyl (meth)acrylates and aminoalkyl (meth)acrylamides;
monomers comprising at least one secondary tertiary or quaternary amine function, or a heterocylcic group containing an aromatic or nonaromatic nitrogen atom, vinylamine or ethyleneimine;
diallyldialkylammonium salts;
alone or as blends, or the corresponding salts, and also macromonomers derived from such monomers.

These monomers have been described previously, and reference may be made to the lists.

As regards the anionic hydrophilic monomers from which these inner amphiphilic polymers may be obtained, mention may be made, for example, of monomers comprising at least one carboxylic, sulfonic, sulfuric, phosphonic, phosphoric or sulfosuccinic function, or the corresponding salts.

More particularly, the monomers are chosen from:
linear, branched, cyclic or aromatic monocarboxylic or polycarboxylic acids, N-substituted derivatives of such acids; polycarboxylic acid monoesters comprising at least one ethylenic unsaturation;
linear, branched, cyclic or aromatic vinylcarboxylic acids;
amino acids comprising one or more ethylenic unsaturations;
   alone or as blends, precursors thereof, sulfonic or phosphonic derivatives thereof, and also macromonomers derived from such monomers; the monomers or macromonomers possibly being in the form of salts.

Examples of anionic monomers that may be mentioned, without wishing to be limited thereto, include:
acrylic acid, methacrylic acid, fumaric acid, itaconic acid, citraconic acid, maleic acid, acrylamidoglycolic acid, 2-propene-1-sulfonic acid, methallylsulfonic acid, styrenesulfonic acid, α-acrylamidomethylpropanesulfonic acid, 2-sulfoethylene methacrylate, sulfopropylacrylic acid, bis(sulfopropyl)acrylic acid, bis(sulfopropyl)methacrylic acid, sulfatoethylmethacrylic acid, hydroxyethylmethacrylic acid phosphate monoester, and also the alkali metal salts, for instance the sodium or potassium salt, or the ammonium salts;
vinylsulfonic acid, vinylbenzenesulfonic acid, vinylphosphonic acid, vinylidenephosphoric acid, vinylbenzoic acid, and also the alkali metal salts, for instance the sodium or potassium salts, or the ammonium salts;
N-methacryloylalanine or N-acryloylhydroxyglycine;
   alone or as blends, and also macromonomers derived from such monomers.

It would not constitute a departure from the, context of the present invention to use monomers which are precursors of those that have just been mentioned. In other words, these monomers contain units that, once incorporated into the polymer chain, may be converted, especially via a chemical treatment such as hydrolysis, to regenerate the abovementioned anionic species. For example, the totally or partially esterified monomers of the abovementioned monomers may be used in order thereafter to be totally or partially hydrolyzed.

Preferably, the inner amphiphilic polymers have a weight-average molar mass of less than or equal to 100 000 g/mol, more particularly between 1000 and 50 000 g/mol and preferably between 1000 and 20 000 g/mol. It is pointed out that the weight-average molar masses indicated above are theoretical molar masses, evaluated as a function of the respective amounts of monomers introduced during the preparation of said polymers.

Preferably, an amphiphilic block polymer of nonionic type is used.

As examples of amphiphilic block polymers that are suitable for use in the invention, mention may be made of polyhydroxystearate-polyethylene glycol-polyhydroxystearate triblock polymers (the products of the Arlacel range from ICI are an example thereof), polymers containing polyalkylpolyether-grafted polydimethylsiloxane blocks (for instance the products of the Tegopren brand name sold by Goldschmidt).

According to a second possibility, at least one cationic surfactant is used as inner surfactant. In the case of this second variant, the inner surfactant does not satisfy Bancroft's rule mentioned previously. Specifically, the cationic surfactant is soluble in the dispersed phase rather than in the continuous phase of the inverse emulsion.

Among the cationic surfactants that are suitable, it is especially possible to use aliphatic or aromatic fatty amines, aliphatic fatty amides and quaternary ammonium derivatives (Rhodoquat RP50 from Rhodia Chimie).

Finally, a third variant of the invention consists in combining the two possibilities that have just been described.

Irrespective of the variant adopted, the total amount of inner surfactant and/or inner amphiphilic polymer more particularly represents from 0.1% to 10% by weight and preferably from 2% to 10% by weight relative to the inner aqueous phase.

In addition, the inverse emulsion more particularly has an inner aqueous phase/organic phase weight ratio of between 10/90 and 90/10. Preferably, an aqueous phase/organic phase weight ratio is between 30/70 and 80/20.

As has already been mentioned, the combination of inner aqueous phase and organic phase is dispersed in an aqueous phase.

Said aqueous phase may optionally comprise at least one active material.

Reference may be made to that which has been stated previously regarding the nature and amount of aqueous phase-soluble active material.

In addition, the emulsion that has just been described may optionally comprise at least one outer surfactant and/or at least one outer amphiphilic polymer.

In this case also, that which has been described regarding the nature of the surfactant/amphiphilic polymer, the various variants and the respective contents, in the context of the aqueous phase of a simple emulsion, is valid in the case of this variant, and reference may be made thereto.

In addition, the outer aqueous phase may comprise at least one additive, one of the roles of which is to equilibrate the osmotic pressures of the outer aqueous phase and of the inner aqueous phase of the multiple emulsion. Among the additives that may be envisioned, mention may be made of salts chosen from alkali metal or alkaline-earth metal halides (for instance sodium chloride or calcium chloride), at least one alkali metal or alkaline-earth metal sulfate (for instance sodium sulfate) or mixtures thereof; sugars (for example glucose), or polysaccharides (especially dextran), or mixtures thereof. The outer aqueous phase may comprise a combination of all these additives.

The concentrations of salt, sugar and/or polysaccharide are such that the osmotic pressures of the outer and inner aqueous phases are equilibrated.

According to one advantageous variant of the present invention, the aqueous phase of the emulsion may comprise at least one thickening polymer. The effect of this polymer is to prevent the creaming and/or sedimentation of the final emulsion.

By way of illustration, thickening polymers extracted from plants and optionally modified, such as carrageenans, alginates, carboxymethylcelloses, methylcelluloses, hydroxypropylcelluloses, hydroxyethylcelluloses and gellans may be used.

It is likewise possible to use thickening polymers of the type such as polysaccharides of animal, plant or bacterial origin; nonlimiting examples that may be mentioned include xanthan gum, guar and derivatives (for instance hydroxypropyl guar) and polydextroses, or combinations thereof.

When it is present, the content of thickening polymer is more particularly between 0.1% and 2% by weight relative to the aqueous phase and preferably between 0.1% and 0.5% by weight relative to the aqueous phase.

The weight ratio of the assembly (inner aqueous phase and organic phase)/aqueous phase represents 0.1%/99.9% to 90/10.

According to this embodiment of the invention, in which the emulsion is in the form of a multiple emulsion, the subject of a second variant is multiple emulsions comprising an inner organic phase dispersed in the organic phase, the combination of the inner organic phase and of the organic phase being dispersed in the aqueous phase. Furthermore, the inner organic phase and the organic phase comprise at least one inner surfactant and/or at least one inner amphiphilic polymer.

That which has been stated previously in the text regarding the nature of the organic phases remains valid and will not be repeated here.

It is simply pointed out that the organic phase and the inner organic phase are chosen such that, they are immiscible or sparingly miscible. Thus, the nature of these two phases is chosen such that the solubility of one in the other does not exceed 10% by weight at a temperature of between 20° C. and the emulsion preparation temperature.

Preferably, according to this embodiment, one of the two organic phases is a silicone phase. Preferably, the silicone phase is the organic phase.

Moreover, optionally, at least one of the organic and inner organic phases may comprise at least one active material.

The description relating to the nature and content of active materials of this type, included in the composition of the organic phase of a simple emulsion, may be repeated here.

The inner organic phase/organic phase emulsion comprises at least one inner stabilizing polymer.

More specifically, said stabilizing polymer satisfies the conditions of Bancroft's rule defined previously.

It is pointed out for purely indicative purposes that the number-average molar mass of the copolymer is less than or equal to 100 000 g/mol and more particularly between 1000 and 50 000 g/mol. The number-average molar mass is given here with an absolute value, and may advantageously be determined by steric exclusion chromatography coupled to the MALLS method.

Advantageously, if each of the blocks of the copolymer represented a polymer (same size and composition as the blocks), then the monomers constituting each of the blocks would be chosen such that each polymer is soluble, under the temperature and concentration conditions mentioned above, either in the inner organic phase (for the polymer derived from the blocks that are soluble in this phase) or in the organic phase (for the polymer derived from the blocks that are soluble in this phase).

More particularly, the fraction of the copolymer that is soluble in the silicone phase is derived from a polysiloxane that may be chosen from the silicones listed in the context of the compounds that may be used as organic silicone phase.

Preferably, the polysiloxane bears reactive functions, for instance —OH or —NH₂ functions, inter alia.

As regards the fraction that is soluble in the nonsilicone organic phase, this is preferably derived from the polymerization of at least one monomer chosen from the following monomers:
linear, branched, cyclic or aromatic monocarboxylic or polycarboxylic acid esters comprising at least one ethylenic unsaturation;
α,β-ethylenically unsaturated nitriles, vinyl ethers, vinyl esters, vinylaromatic monomers, vinyl halides or vinylidene halides;
linear or branched, aromatic or nonaromatic hydrocarbon-based monomers comprising, at least one ethylenic unsaturation;
   alone or as blends, and also macromonomers derived from such monomers.

It should be noted that the fraction of the copolymer that is soluble in this nonsilicone phase may be obtained from the abovementioned monomers, combined with monomers of different chemical nature, for instance nonionic or ionic hydrophilic monomers.

According to one advantageous embodiment, the copolymer is a linear block copolymer preferably comprising at least three blocks.

The copolymers that may be used may be advantageously obtained via a free-radical route, preferably a controlled free-radical route.

Preferably, such polymers may be obtained by performing a preparation process via heat activation of silicone and organic hybrid copolymers comprising units (I):
RₓU_{y}SiO_{[4-(x+y)]/2} (I)
in which:
   x is equal to 0, 1, 2 or 3, y is equal to 0, 1, 2 or 3 with 2≦(x+y)≦3 and y is other than 0 for at least one of the units of the hybrid copolymer,
   the symbols R, which may be identical or different, represent:
      a linear or branched alkyl radical containing 1 to 8 carbon atoms, optionally substituted with at least one halogen, preferably fluorine, the alkyl radicals preferably being methyl, ethyl, propyl, octyl and 3,3,3-trifluoropropyl,
      a cycloalkyl radical containing between 5 and 8 ring carbon atoms, which is optionally substituted,
      an aryl radical containing between 6 and 12 carbon atoms, which may be substituted, preferably phenyl or dichlorophenyl,
      an aralkyl portion having an alkyl portion containing between 5 and 14 carbon atoms and an aryl portion containing between 6 and 12 carbon atoms, optionally substituted on the aryl portion with halogens, alkyls and/or alkoxyls containing 1 to 3 carbon atoms,
   the symbols U, which may be identical or different, represent (II): 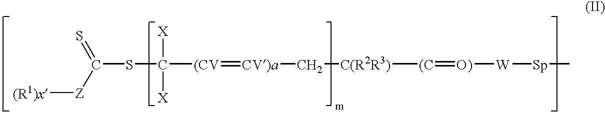
in which:
   x′=1, 2, 3 or 4 as a function of the valency of Z,
   Z, which may be identical or different, represent a carbon, sulfur, oxygen, phosphorus or nitrogen atom and/or a free valency,
R¹, which may be identical or different, represent:
   an alkyl, acyl, aryl, alkene or alkyne group (i), which is optionally substituted,
   a saturated or unsaturated, optionally substituted and/or aromatic carbon-based ring (ii), and/or
   a saturated or unsaturated, optionally substituted heterocycle (iii),
   these radicals (i), (ii) and (iii) possibly being advantageously substituted with: substituted phenyl groups, substituted aromatic groups, or groups as follows: alkoxycarbonyl, aryloxycarbonyl (—COOR⁵), carboxy (—COOH), acyloxy (—O₂CR⁵), carbamoyl (—CONR⁵₂), cyano (—CN), alkylcarbonyl, alkylarylcarbonyl, arylcarbonyl, arylalkylcarbonyl, phthalimido, maleimido, succinimido, amidino, guanidimo, hydroxyl (—OH), amino (—NR⁵₂), halogen, allyl, epoxy, alkoxy (—OR⁵), S-alkyl, S-aryl, groups of hydrophilic or ionic nature, such as alkali metal salts of carboxylic acids, alkali metal salts of sulfonic acid, polyalkylene oxide (PEO, PPO) chains, cationic substituents (quaternary ammonium salts), R⁵, which may be identical or different, representing an alkyl or aryl group, and/or a polymer chain,
a group (iv) of formula —CₙF₍₂ₙ₊₁₎ with n, being between 1 and 20,
a group (v) having the following formula: 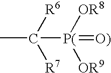
in which:
   R⁶ and R⁷, which may be identical or different, are chosen from a halogen, —NO₂, —SO₃R¹⁰, —NCO, —CN, —OR¹⁰, —SR¹⁰, —N(R¹⁰)₂, —COOR¹⁰, —O₂CR¹⁰, —CON(R¹⁰)₂, —NCO(R¹⁰)₂ and —CₙF₍₂ₙ₊₁₎ group with n between 1 and 20, and preferably equal to 1;
   with R¹⁰ representing:
   a hydrogen atom,
   or an alkyl, alkenyl, alkynyl, cycloalkenyl, cycloalkynyl, alkaryl, aralkyl, heteroaryl or aryl radical optionally fused to an aromatic or nonaromatic heterocycle; these radicals possibly being substituted with:
      one or more groups, which may be identical or different, chosen from halogen atoms, ═O, ═S, —OH, alkoxy, SH, thioalkoxy, NH₂, monoalkylamino or dialkylamino, —CN, COOH, ester, amide and CₙF₍₂ₙ₊₁₎ and/or optionally interrupted with one or more atoms chosen from O, S, N and P;
      or a heterocyclic group optionally substituted with one or more groups as defined above;
      or R⁶ and R⁷ form, together with the carbon atom to which they are attached, a group ═O, ═S, a hydrocarbon-based ring or a heterocycle;
      and R⁸ and R⁹, which may be identical or different, represent a group as defined above for R¹⁰; or together form a C₂-C₄ hydrocarbon-based chain optionally interrupted with a hetero atom chosen from O, S and N;
   V and V′, which may be identical or different, represent: H, an alkyl group or a halogen,
   X and X′, which may be identical or, different, represent H, a halogen or a group R⁴, OR⁴, O₂COR⁴, NHCOH, OH, NH₂, NHR⁴, N(R⁴)₂, (R⁴)₂N⁺O⁻, NHCOR⁴, CO₂H, CO₂R⁴, CN, CONH₂, CONHR⁴ or CONR⁴₂, in which R⁴ is chosen from alkyl, aryl, aralkyl, alkaryl, alkene and organosilyl groups, optionally, perfluorinated and optionally substituted with one or more carboxyl, epoxy, hydroxyl, alkoxy, amino, halogen or sulfonic groups,
   R² and R³, which may be identical or different, represent:
      an alkyl, acyl, aryl, alkene or alkyne group (i), which is optionally substituted,
      a saturated or unsaturated, optionally substituted and/or aromatic carbon-based ring (ii),
      a saturated or unsaturated, optionally substituted heterocycle (iii),
      a hydrogen atom or groups from the following: alkoxycarbonyl, aryloxycarbonyl (—COOR⁵), carboxyl (—COOH), acyloxy (—O₂CR⁵); carbamoyl (—CONR⁵₂), cyano (—CN), alkylcarbonyl, alkylarylcarbonyl, arylcarbonyl, arylalkylcarbonyl, phthalimido, maleimido, succinimido, amidino, guanidimo, hydroxyl (—OH), amino (—NR⁵₂), halogen, allyl, epoxy, alkoxy (—CR⁵), S-alkyl, S-aryl, groups of hydrophilic or ionic nature such as alkali metal salts of carboxylic acids, alkali metal salts of sulfonic acids, polyalkylene oxide (PEO or PPO) chains, cationic substituents (quaternary ammonium salts), R⁵, which may be identical or different, representing an alkyl or aryl group, and/or a polymer chain,
      the radicals (i), (ii) and (iii) possibly being advantageously substituted with: substituted phenyl groups, substituted aromatic groups or groups from the following: alkoxycarbonyl, aryloxycarbonyl (—COOR⁵), carboxyl (—COOH), acyloxy (—O₂CR⁵), carbamoyl (—CONR⁵₂), cyano (CN), alkylcarbonyl, alkylarylcarbonyl, arylcarbonyl, arylalkylcarbonyl, phthalimido, maleimido, succinimido, amidino, guanidimo, hydroxyl (—OH), amino (—NR⁵₂), halogen, allyl, epoxy, alkoxy (—OR⁵), S-alkyl, S-aryl, groups of hydrophilic or ionic nature such as alkali metal salts of carboxylic acids, alkali metal salts of sulfonic acids, polyalkylene oxide (PEO or PPO) chains, cationic substituents (quaternary ammonium salts), R⁵, which may be identical or different, representing an alkyl or aryl group, and/or a polymer chain,
   W, which may be identical or different, represent a divalent radical chosen from —O—, —NR⁴—, —NH— and —S—,
   Sp, which may be identical or different, represents a coupling unit consisting of a divalent organic radical of formula —(CH₂)_{x′″}—, in which x′″ is between 1 and 20, this radical possibly being substituted with and/or containing at least one hetero atom,
   a=0 or 1,
   m≧1, and, when m>1, the repeating unit moieties of index m are identical or, different,
      and to a process in which the following are placed in contact:
at least one ethylenically unsaturated monomer of formula (III):
   CXX′(═CV-CV′)ₐ=CH₂,
a precursor silicone compound comprising units, which may be identical or different, of formula (IV):
   RₓU′_{y}SiO_{[4-(x+y)]/2}
in which:
   R, x and y correspond to the values given above,
and the monovalent radical U is according to formula (V) below: 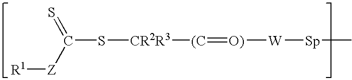
and a free-radical polymerization initiator.

The free-radical polymerization initiator may be chosen from the initiators conventionally used in free-radical polymerization. It may be, for example, one of the following initiators:
hydrogen peroxides such as: tert-butyl hydroperoxide, cumene hydroperoxide, t-butyl peroxyacetate, t-butyl peroxybenzoate, t-butyl peroxyoctoate, t-butyl peroxyneodecanoate, t-butyl peroxyisobutytate, lauroyl peroxide, t-amyl peroxypivalate, t-butyl peroxypivalate, dicumyl peroxide, benzoyl peroxide, potassium persulfate, ammonium persulfate,
azo compounds such as: 2,2′-azobis(isobutyronitrile), 2,2′-azobis(2-butanenitrile), 4,4′-azobis(4-pentanoic acid), 1,1′-azobis(cyclohexanecarbonitrile), 2-(t-butylazo)-2-cyanopropane, 2,2′-azobis[2-methyl-N-(1,1)-bis(hydroxymethyl)-2-hydroxyethyl]propionamide, 2,2′-azobis(2-methyl-N-hydroxyethyl]propionamide, 2,2′-azobis(N,N′-dimethyleneisobutyramidine)dichloride, 2,2′-azobis(2-amidinopropane)dichloride, 2,2′-azobis(N,N′-dimethyleneisobutyramide), 2,2′-azobis(2-methyl-N-[1,1-bis(hydroxymethyl)-2-hydroxyethyl]propionamide), 2,2′-azobis(2-methyl-N-[1,1-bis(hydroxymethyl)ethyl]-propionamide, 2,2′-azobis[2-methyl-N-(2-hydroxyethyl)propionamide], 2,2′-azobis(isobutyramide)dihydrate,
redox systems comprising combinations such as: mixtures of hydrogen peroxide, alkyl peroxide, peresters, percarbonates and the like and any iron salts, titanium salts, zinc formaldehydesulfoxylate or sodium formaldehydesulfoxylate, and reducing sugars; ammonium or alkali metal persulfates, perborate or perchlorate in combination with an alkali metal bisulfite, such as sodium metabisulfite, and reducing sugars; alkali metal persulfates in combination with an arylphosphinic acid, such as benzenephosphonic acid and the like, and reducing sugars.

The amount of initiator to be used is determined such that the amount of radicals generated is not more than 20 mol % relative to the amount of silicone precursor compound (IV) and preferably not more than 5 mol %.

Ethylenically unsaturated monomers that are used include those mentioned previously to define the fraction of the copolymer that is soluble in the nonsilicone organic phase.

It is furthermore specified that butadiene and chloroprene correspond to the case in which a=1 in formulae (I) and (III).

For the preparation of the hybrid copolymers of formula (I) for which X=H and X′=NH₂, the ethylenically unsaturated mono ers preferably used are vinylamine amides, for example vinylformamide or vinylacetamide. The copolymer obtained is then hydrolyzed at acidic or basic pH.

For the preparation of the hybrid copolymers of formula (I) for which X=H and X′=OH, the ethylenically unsaturated monomers that are preferably used are vinyl esters of carboxylic acid, such as, for example, vinyl acetate. The copolymer obtained is then hydrolyzed at acidic or basic pH.

The types and amounts of copolymerizable monomers used according to the present invention vary depending on the particular final application for which the hybrid copolymer is intended.

According to a first preferred variant, the silicone and organic hybrid copolymer consists of a linear silicone skeleton comprising from 1 to 300 units and preferably 1 to 200 units of formula (I), bearing from 1 to 50 and preferably 1 to 10 radicals U.

According to a second variant, at least one of the monovalent radicals U′ is preferably of formula (VI): 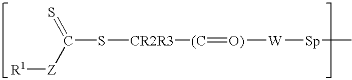
in which R² and R³, which may be identical or different, represent a hydrogen atom or a cyano, alkoxycarbonyl, alkyl, acyl, aryl, alkene or alkyne group, which is optionally substituted; and W has an identical definition to that given above, and is preferably equal to an oxygen atom. Examples that will be mentioned include the silicone precursors in which U′ corresponds to:

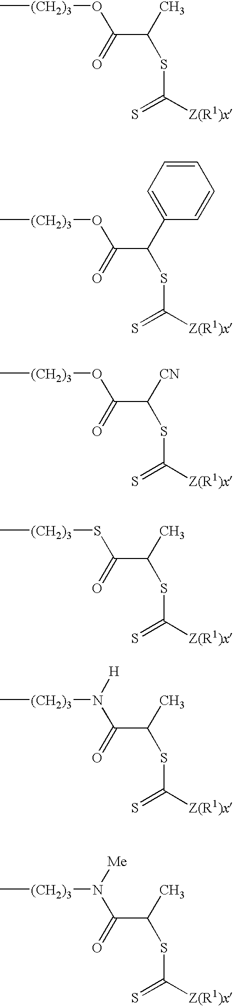

According to a third variant of the invention, at least some of the monovalent radicals U′ of the silicone precursor(s) (IV) and thus at least some of the groups U of the hybrid copolymer obtained are such that Z is an oxygen atom and/or a sulfur atom.

According to a fourth variant in addition to the units of formula (I), the silicone and organic hybrid copolymer according to the invention may comprise units RₓU_{y}F_{z}SiO_{[4-(x+y+z)]/2} (XIV) in which:
x is equal to 0, 1, 2 or 3, y is equal to 0, 1, 2 or 3 and z is equal to 1, 2 or 3 with 2≦(x+y+z)≦3, and
F is a group bearing at least one function such as hydroxyl, alkoxy, thiol, amine, epoxy and/or polyether.

These groups F may optionally provide complementary and/or additional properties to the hybrid copolymers prepared according to the process of the invention. They may especially be initially contained in the silicone precursor of formula (IV).

In addition to the hybrid copolymers with homopolymer organic segments, the process which has just been described makes it possible to prepare hybrid polymers bearing organic groups in blocks (i.e. multiblocks). To do this, the process consists in repeating the implementation of the preparation process described above, using:
monomers that are different than in the previous implementation, and
in place of the precursor silicone compound (II), the hybrid copolymer comprising the block units (I), obtained from the above implementation.

According to this process for preparing block copolymers, when it is desired to obtain copolymers containing homogeneous blocks without a composition gradient, and if all the successive polymerizations are performed in the same reactor, it is essential that all the monomers used in one step should have been consumed before the polymerization of the next step begins, and thus before the new monomers are introduced.

As for the process for polymerizing a monoblock copolymer, this process for polymerizing block copolymers has the advantage of producing block copolymers with a low polydispersity index. It also allows the molecular mass of the block polymers to be controlled.

The precursor silicone compound of general formula (IV) used in the process for preparing the hybrid copolymers according to the invention may be obtained by reacting:
(i) a silicone comprising units of formula (VII):
   RₓU″_{y}SiO_{[4-(x+y)]/2}
in which the monovalent radical U″ is according to formula (VIII) below: 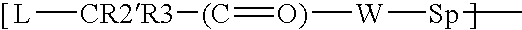
in which:
   W and Sp are definitions identical to those given above,
   L is a nucleophilic group, for example: Br⁻, Cl⁻, I⁻, OTs⁻, OMs⁻, (C₆H₆)—(C═O)—O⁻, (CH₃)—(C═O)—O⁻, (CF₃)—(C═O)—O⁻,
(ii) with a compound chosen from those of general formula (IX), (X) or (XI) below: 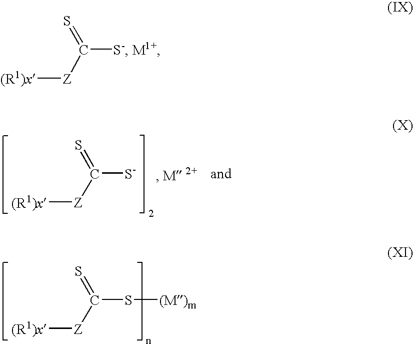
in which:
   M¹⁺ represents K⁺, Na⁺, NR₄⁺ or PR₄⁺, R having a definition similar to that given for R in formula (I),
   M″²⁺ represents an alkaline-earth metal such as Ca²⁺, Ba²⁺ or Sr²⁺⁺,
   M′″ represents Zn, Cd, m is equal to 1 or 2, n is equal to 1, 2, 3 or 4 and preferably m is equal to 1 and n is equal to 2.

This silicone of formula (VII) may especially be obtained from (i) a silicone comprising units of formula (XII): RₓU′″_{y}SiO_{[4-(x+y)]/2} in which the monovalent radical U′″ is of formula (XIII): -Sp-WH and (ii) of a compound of formula:

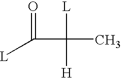

The polymerization may be performed in bulk, in solution or in emulsion. It is preferably performed in emulsion.

The process is preferably performed semi-continuously.

The temperature may range between room temperature and 150° C. depending on the nature of the monomers used.

In general, during the polymerization, the instantaneous copolymer content relative to the instantaneous amount of monomer and copolymer is between 50% and 99% by weight, preferably between 75% and 99% and even more preferably between 90% and 99%. This content is maintained, in a known manner, by controlling the temperature and the rate of addition of the reagents and of the polymerization initiator.

Finally, the process is generally performed in the absence of a UV source.

It should be noted that it may be advantageous to chemically modify the xanthate end groups of the copolymer obtained, by using any method known to those skilled in the art, for instance a hydrolysis step.

The process and the polymers obtained via this process are described in French patent application 00 09722 filed on Jul. 25, 2000.

Other methods for synthesizing copolymers of this type may be used, especially the method described in International patent applications WO 00/71606 and WO 00/71607.

According to one particular embodiment of the invention, the amount of inner stabilizing copolymer represents from 0.5% to 10% by weight of the inner organic phase and preferably between 1% and 4% by weight relative to the same reference.

The weight ratio of inner organic phase relative to the organic phase is more particularly between 10/90 and 90/10 and preferably between 30/70 and 50/50.

As regards the weight ratio of the combination (inner organic phase and organic phase)/aqueous phase, this represents 0.1/99.9 to 90/10.

The aqueous phase, in which is dispersed the organic phase comprising the inner organic phase, comprises at least one outer surfactant and/or outer stabilizing polymer and optionally at least one active material.

That which has been detailed in the context of the preceding variant remains valid here and reference may be made thereto.

The simple emulsion or the inner aqueous phase/organic phase emulsion or the inner organic phase/aqueous phase emulsion is prepared by performing the standard methods.

Thus, in the case of simple emulsions, a first mixture comprising the compound used as organic phase and optionally an active material is prepared, on the one hand, and the aqueous mixture optionally comprising the active material, optionally the outer surfactant/outer amphiphilic polymer and optionally the block copolymer is then prepared, on the other hand. The mixture constituting the dispersed phase is then added to the mixture constituting the continuous phase of the emulsion.

In the case of multiple emulsions, the “inner” emulsion is first prepared.

For example, in the case of an emulsion of an inner aqueous phase and of an organic phase, two mixtures are prepared, the first which is an aqueous mixture optionally comprising the active material, optionally the inner surfactant (if it is more particularly chosen from cationic surfactants) and optionally at least one additive (salt/sugar/polysaccharide), and then a second mixture comprising the compound constituting the organic phase, optionally the active material, and optionally the inner surfactant/inner stabilizing polymer; and the first mixture is then added to the second mixture.

In the context of an emulsion of an inner organic phase in an organic phase, two mixtures are prepared in this case also; the first constituting the inner organic phase comprising the compound constituting the organic phase and optionally an active material; and then a second mixture comprising the compound constituting said organic phase, optionally an active material, and the inner stabilizing polymer; the first mixture is then introduced into the second.

Once the inner emulsion has been prepared, said emulsion is emulsified in the aqueous phase (which will be the outer aqueous phase of the multiple emulsion).

Thus, the mixture constituting the aqueous phase is prepared and optionally comprises an active material, optionally an outer surfactant/outer amphiphilic polymer, optionally an additive (salt/sugar/polysaccharide), optionally the thickening polymer, and optionally the block copolymer.

Specifically, according to a first possibility, the block copolymer is incorporated during the actual preparation of the emulsion. In other words, the block copolymer is added during the preparation of the emulsion of the organic phase and of the aqueous phase; the organic phase optionally comprising a dispersed inner aqueous phase or a dispersed inner organic phase.

More specifically, said block copolymer is present in the mixture constituting the aqueous phase (aqueous phase of the simple emulsion or outer aqueous phase of the multiple emulsion).

It is indicated that, in this case, the presence of an outer surfactant and/or outer amphiphilic polymer may not be necessary, even though, advantageously, they are present in the aqueous phase

According to this possibility, the organic phase (optionally comprising the inner aqueous or organic phase)/aqueous phase weight ratio is advantageously between 10/90 and 90/10.

Furthermore, in the case where a thickening polymer is used, it is preferable to incorporate it once the emulsion has been obtained, preferably in the form of an aqueous solution, the water content of which will be calculated such that the organic phase (optionally comprising the inner aqueous or organic phase)/aqueous phase weight ratio is within the range 0.1/99.9 to 90/10.

According to a second possibility, the block copolymer is added to the emulsion, irrespective of the variant under consideration, once the preparation of the emulsion itself has been performed, i.e. the preparation of the emulsion of the organic phase and of the aqueous phase; the organic phase optionally comprising a dispersed inner aqueous phase or a dispersed inner organic phase.

In this case, the outer surfactant and/or outer amphiphilic polymer is present in the aqueous phase of the simple or multiple emulsion.

In the context of this possibility, said block copolymer is added to the emulsion, advantageously, in the form of an aqueous solution; the amount of aqueous phase used being calculated as a function of the amount of aqueous phase added during the preparation of the emulsion, and such that the organic phase (optionally comprising the inner aqueous, or organic phase)/aqueous phase weight ratio is within the range 10/90 to 90/10.

It is furthermore pointed out that the block copolymer may be introduced at the time of preparation of the final aqueous formulation. In such a case, the organic phase (optionally comprising the inner aqueous or organic phase)/aqueous phase weight ratio is within the range 0.1/99.9 to 10/90.

These mixing operations during the various steps for the preparation of the simple or multiple emulsion are usually performed with stirring.

Moreover, conventionally, the emulsion is generally prepared at a temperature greater than or equal to the melting point of the organic phase(s) present. As a guide, the emulsion preparation temperature is between 20 and 80° C.

The emulsions that have just been detailed may be used especially in the fields of skin and/or hair treatment, the treatment of textiles (especially for washing textiles), the treatment of plants (plant protection sector) or the treatment of metal.

One of the advantages of emulsions comprising the block copolymer in aqueous formulations comprising, anionic surfactants (shampoos, shower gels, textile washing formulations, etc.) is that they allow an emulsion to be deposited onto a surface, using precipitation-flocculation mechanisms during the dilution of the formulation.

One particularly advantageous embodiment of the invention consists in that:
the emulsion to be deposited represents a constituent component of an aqueous liquid detergent formulation for washing articles made of textile fiber, comprising at least one anionic surfactant,
the emulsion is a simple emulsion, the organic phase of which comprises at least one organic or organosilicon hydrophobic active material,
the emulsion is intended to be deposited on the surface of articles made of textile fiber during a washing operation.

One particularly advantageous subject of the invention is thus directed toward a particular embodiment thereof, which consists of the use, in an aqueous liquid detergent formulation for washing articles made of textile fiber, comprising at least one anionic surfactant and at least one hydrophobic organic or organosilicon active material as an emulsion in an aqueous phase, of at least one block copolymer comprising at least one nonionic hydrophobic block and at least one cationic block under the conditions of the operation for washing said articles, as an agent to assist the deposition, during the washing operation, of said active material in emulsion on the surface of said articles.

Another subject of the invention is directed toward a process for assisting, during an operation for washing articles made of textile fiber using an aqueous liquid detergent formulation comprising at least one anionic surfactant and at least one hydrophobic organic or organosilicon active material in emulsion in an aqueous phase, the deposition of said active material in emulsion onto the surface of said articles, this process consisting in adding to said detergent formulation at least one block copolymer comprising at least one nonionic hydrophobic block and at least one cationic block under the conditions of the washing operation.

Preferably, the aqueous liquid detergent composition for washing articles made of textile fiber may comprise from 5% to 50% by mass of at least one anionic surfactant; the amount of hydrophobic organic or organosilicon active material may represent from 0.01% to 10%, preferably from 0.1% to 5% and more preferably from 0.2% to 4% of the mass of the formulation; the amount of block copolymer may represent from 0.1% to 25%, preferably from 1% to 15% and more particularly from 3% to 11% of the mass of the hydrophobic organic or organosilicon active material.

The amount of detergent formulation that may be used to perform the washing operation may be from about 1 to 10 grams per liter of washing water; the active material in emulsion flocculates by precipitation, especially on dilution.

The organic or organosilicon active material in emulsion is preferably chosen from oils or waxes of plant origin, fatty acid esters, mineral oils, paraffin waxes, nonionic silicone oils or waxes, aminosilicone oils, in particular silicone oils containing hindered piperidyl function(s). This type of active material allows the textile surfaces to be given creaseproof properties and/or easy-ironing properties and/or antisoiling properties and/or better abrasion resistance.

The active material present in the organic phase of the emulsion may also be present at the same time as another hydrophobic detergency constituent that is soluble in said active material; mention may be made especially of hydrophobic fragrances, optical brighteners or biocides.

The detergent formulation, the active material emulsion of which constitutes one of the essential components, may also contain along with the anionic surfactant(s) other additives usually used in detergent compositions, especially liquid detergent compositions, for washing articles made of textile fiber.

Mention may be made especially of nonionic surfactants especially mineral detergency adjuvants (“builders”) (polyphosphates, silicates, carbonates, zeolites, etc.) or organic detergency adjuvants (“builders”) (water-soluble polyphosphonates, polycarboxylates, polycarboxylic acids, polyacetic acid salts, etc.), antisoiling agents (cellulose derivatives, polyester copolymers based on ethylene terephthalate units and polyoxyethylene terephthalate units, sulfonated polyester oligomers or copolymers, etc.), antiredeposition agents (ethoxylated monoamines or polyamines, carboxymethylcelluloses, polyvinylpyrrolidones, etc.), iron- or magnesium-chelating agents (aminocarboxylates, aminophosphonates, dihydroxydisulfobenzene, etc.), polymeric dispersants (water-soluble salts of polycarboxylic acids with a molecular mass of about from 2000 to 100 000 g/mol, polyethylene glycols with a molecular mass of about from 5000 to 75 000 g/mol, etc.), fluorescers, foam suppressants, softeners (clays, etc.), enzymes, buffers, fragrances, pigments, etc.

When the emulsions comprising the block copolymer are used in formulations free of anionic surfactants, said emulsions may be deposited by means of electrostatic interactions onto surfaces having overall an anionic charge potential.

In the case where the emulsions are used in compositions intended for treating the skin and/or the hair, they may be introduced into aqueous formulations intended to be rinsed and especially comprising from 3% to 50% by weight of ionic or nonionic surfactants.

These formulations comprise additives that are standard in the field, for instance UV-screening agents, dispersants, sequestering agents, emollients, humectants, conditioners, fixing resins, plasticizers, dyes, pigments, fragrances bactericides, etc.

The examples that follow are given for illustrative purposes.

### EXAMPLE 1

Synthesis of a polybutyl acrylate 2000-poly(2-dimethylaminoethyl acrylate)4000 Diblock Copolymer

155.7 g of ethanol, 10.41 g of O-ethyl-S-(1-methoxycarbonyl)ethyl)xanthate (CH₃CHCO₂CH₃) S(C═S)OEt and 100 g of butyl acrylate are placed in a 1 liter two-necked glass round-bottomed flask equipped with a condenser and a magnetic stirrer and maintained under argon. The solution is brought to a temperature of 70° C. and a solution of 3.28 g of azobisisobutyronitrile (AIBN) in 9.85 g of acetone and 4.92 g of ethanol is added. Three hours after this addition, a solution of 1.64 g of AIBN in 4.93 g of acetone and 2.46 g of ethanol is added.

At this stage, a sample is taken. The number-average molar mass (Mn) is measured by steric exclusion chromatography in dimethylformamide, coupled to a multiangle light-scattering detector (GPC-MALLS).

Mn=2200 g/mol.

A solution of 200 g of 2-dimethylaminoethyl acrylate in 280 g of ethanol is added over three hours to the reaction medium. Two hours after the end of this introduction, a solution of 2.46 g of AIBN in 7.39 g of acetone and 3.69 g of ethanol is added. Two hours later, this same AIBN solution is added again. After this last addition, the reaction is continued for two hours and then stopped by cooling the reaction medium to room temperature.

### EXAMPLE 2

Synthesis of a poly(2-trimethylaminoethyl acrylate methyl sulfate) 40 000 (Cationic)-polybutyl acrylate 3000 Diblock Copolymer

A solution composed of 236 g of water, 75 g of ethanol and 308 g of a solution of 2-trimethylaminoethyl acrylate methyl sulfate (i.e. 2-dimethylaminoethyl acrylate quaternized with methylsulfate) at a concentration of 80% in water is prepared in a beaker. 150 g of the solution described above and a solution of 1.28 g of O-ethyl-S-(1-methoxycarbonyl)ethyl)xanthate (CH₃CHCO₂CO₃)S(C═S)OEt in 25 g of ethanol, are placed in a 1 liter two-necked glass round-bottomed flask equipped with a condenser and a magnetic stirrer and maintained under argon. 0.834 g of 4,4′-azobis(4-cyanovaleric acid) (ACP) is also added to the reaction mixture. The solution is brought to a temperature of 70° C. 15 minutes after the start of the reaction, the rest of the monomer solution is added continuously over a period of 2.5 hours. At the end of introduction of the monomer solution, a further 0.85 g of initiator in 5 g of water is added to the reaction mixture. The reaction is stopped after an overall polymerization time of 6 hours.

A sample of 18 g of solution is then taken. Analysis by ¹H NMR confirms that the acrylate monomer has been totally polymerized.

18 g of butyl acrylate are added in a single portion to the polymer obtained from the first step, followed by addition of 0.9 g of ACP. After reaction for 3 hours, a further 0.9 g of ACP is added. The reaction is then continued for 5 hours. At the end of reaction, the diblock copolymer is obtained. By simple evaporation of the ethanol on a rotary evaporator, a stable aqueous dispersion of the diblock copolymer is obtained.

### EXAMPLE 3

A poly(2-trimethylaminoethyl acrylate methyl sulfate) 4000 (cationic)-polybutyl acrylate 3000 diblock copolymer is prepared according to a procedure similar to that of example 2.

### EXAMPLE 4

Preparation of the Emulsion A and of the Shampoo SA

27 g of an aqueous solution containing 0.3 g by weight of the block copolymer of example 1 are prepared. The pH of this solution is adjusted to 5. 3 grams of Rhodorsil 48V5000 silicone oil from Rhodia (polydimethylsiloxane oil blocked at each of the ends with a (CH₃)₂HOSiO_{0.5} unit and having a viscosity of 5000 mPa.s at 25° C.) are then added thereto. The mixture is homogenized by a first blending in an Ultra-Turrax blender at 9500 rpm for 5 minutes and then in a microfluidizer (5 times at 500 bar). Emulsion A is obtained.

70 g of an aqueous formulation containing 14 g of sodium lauryl ether sulfate (Empicol ESB3M), 2 g of Tegobetaine T7 and 1.6 g of sodium chloride are prepared. The pH is adjusted to 5. The 30 g of emulsion A are then introduced into the 70 g of the above formulation, with stirring using a frame paddle (300 rpm for 5 minutes). The shampoo SA is obtained.

Preparation of the Emulsion B and of the Comparative Shampoo SB

27 g of an aqueous solution containing 0.3 g by weight of sodium dodecyl sulfate are prepared. The pH of this solution is adjusted to 5. 3 grams of Rhodorsil 48V5000 silicone oil from Rhodia are then added thereto. The mixture is homogenized by a first blending in an Ultra-Turrax blender at 9500 rpm for 5 minutes and then in a microfluidizer (5 times at 500 bar). Emulsion B is obtained.

70 g of an aqueous formulation containing 14 g of sodium lauryl ether sulfate (Empicol ESB3M), 2 g of Tegobetaine T7 and 1.6 g of sodium chloride are prepared. The pH is adjusted to 5. The 30 g of emulsion B are then introduced into the 70 g of the above formulation, with stirring using a frame paddle, (300 rpm for 5 minutes). The shampoo SB is obtained.

Dilution Behavior of Shampoos A and B

The shampoos SA and SB are observed by optical microscopy, in their form as produced and after having been diluted 10-fold with an aqueous solution at pH 5.

Flocculation of the shampoo SA is clearly observed on dilution.

On the other hand, no flocculation of the shampoo SB is observed on dilution.

Deposition

The shampoos SA and SB are used to treat identical locks of hair. The deposition of silicone on the hair after washing and rinsing the locks is measured by X-ray fluorescence. It is found that the deposition of silicone obtained with the shampoo SA is greater than that obtained with the shampoo SB.

### EXAMPLE 5

Preparation of the Emulsion C and of the Comparative Shampoo SC

27 g of an aqueous solution containing 0.3 g by weight of sodium dodecyl sulfate are prepared. The pH of this solution is adjusted to 5.

3 grams of Rhodorsil 48V5000 silicone oil from Rhodia are then added thereto. The mixture is homogenized via a first blending using an Ultra-Turrax blender at 9500 rpm for 5 minutes and then a microfluidizer (5 times at 500 bar). Emulsion C is obtained.

70 g of an aqueous formulation containing 14 g of sodium lauryl ether sulfate (Empicol ESB3M), 2 g of Tegobetaine T7 and 1.6 g of sodium chloride are prepared. The pH is adjusted to 5. The 30 g of emulsion C are then introduced into the 70 g of the above formulation, with stirring using a frame paddle (300 rpm for 5 minutes). The shampoo SC is obtained.

Preparation of the Shampoo SD

69.7 g of an aqueous formulation containing 14 g of sodium lauryl ether sulfate (Empicol ESB3M), 2 g of Tegobetaine T7 and 1.6 g of sodium chloride are prepared. The pH is adjusted to 5. The 30 g of emulsion C are then introduced into the 69.7 g of the above formulation, with stirring using a frame paddle (300 rpm for 5 minutes). 0.3 g of block copolymer from example 2 is then introduced with stirring using a frame paddle (300 rpm for 2 minutes). Shampoo SD is obtained.

Deposition

Shampoos SC and SD are used to treat identical locks of hair. The deposition of silicone on the hair after washing and rinsing the locks is measured by X-ray fluorescence. It is found that the deposition of silicone obtained with shampoo SD is greater than that obtained with shampoo SC.

### EXAMPLE 6

Two aqueous emulsions E1 and E2 of silicone oil are prepared from
10 parts by mass of Rhodorsil 47V100 silicone oil from Rhodia (polydimethylsiloxane oil blocked at each of the ends with a (CH₃)₃SiO_{0.5} unit and having a viscosity of 100 mPa.s at 25° C.)
100 parts of water
and, respectively, 0.5 part (E1) and 1 part (E2) by mass of block copolymer of example 3
   by treating the mixture for 5 minutes in an Ultra-Turrax homogenizer rotating at 9500 rpm.

An emulsion whose size is, respectively, 0.5 μm (E1) and 0.3 μm (E2) is obtained.

For comparative purposes, an emulsion E of silicone oil is prepared from
10 parts by mass of Rhodorsil 47V100 silicone oil
100 parts of water
and 1 part by mass of Symperonic A7 nonionic surfactant (ethoxylated fatty alcohol)
   by treating the mixture for 5 minutes in an Ultra-Turrax homogenizer rotating at 9500 rpm.

An emulsion whose size is 0.25 μm is obtained.

These three emulsions are added, at a rate of 1 part by mass of emulsion, expressed as silicone oil, to 100 parts by mass of an anionic liquid washing product L, the composition of which is as follows:

| Constituents | % by mass |
|---|---|
| Sodium carbonate | 20% |
| Lauryl sulfonate | 15% |
| Nonionic C12 surfactant with 7 EO | 10% |
| Sokalan CP5 from BASF (acrylic acid/maleic | 4% |
| anhydride copolymer, sodium salt) | |
| HEDP (phosphonated sequestering agent) | 0.5% |
| Antifoam | 0.2% |
| Enzyme (mixture of protease, amylase, | 0.5% |
| mannanase, lipase and cellulase) | |
| Fragrance | 0.5% |
| Propylene glycol | 5% |
| Water | qs 100 |

The washing products L1; L2 and L′, respectively corresponding to the addition of emulsions E1, E2 and E′ to the washing product L, are obtained.

Washes are performed using the various washing products L, L′, L1 and L2, in a Tergotometer laboratory machine that is well known in the profession of detergent composition formulators. The machine simulates the mechanical and thermal effects of American pulsator-type washing machines, but, by virtue of the presence of 6 washing drums, it allows series of tests to be performed simultaneously with an appreciable saving in time.

For each wash, two samples of terry toweling cotton cloth and two samples of flat cotton cloth 10×10 cm in size are cut up.

They are washed using the chosen detergent formulation and rinsed 3 times, under the following conditions:
number of test pieces per Tergotometer drum: 3
volume of water: 1 liter
water of French hardness 15° TH (tap water)
chosen washing product concentration: 5 ml/l;
wash temperature: 40° C.
wash time: 20 min
spin speed of the Tergotometer: 100 rpm
rinsing with cold water (about 15° TH)
rinsing time: 5 minutes.

The samples are dried vertically at room temperature.

Ten experts are instructed to perform a manual test of softness of the washed samples of terry toweling cotton cloth and to give each test sample a grade ranging from 1 to 5.

Grade 1 is given to a very coarse fabric.

Grade 5 is given to a very soft fabric (new state).

For each test sample, the mean of the values obtained and the mean of the standard deviations are calculated.

The results obtained are as follows:

| Washing | | | | |
|---|---|---|---|---|
| product | L | L′ | L1 | L2 |
| Softness | 1.4 ± 0.51 | 1.9 ± 0.73 | 3.8 ± 0.63 | 4.3 ± 0.67 |

It is found that the block copolymer of example 3 is particularly suitable for improving the deposition of silicone oil as an emulsion onto the surface of the cotton, which is reflected in real terms by greater provision of softness.

1-21. (Canceled)

22. A process to assist the deposition of an emulsion on a surface, under the conditions of use of said emulsion, comprising the steps of adding to said emulsion an efficient amount of at least one block copolymer comprising at least one nonionic hydrophobic block and at least one block containing cationic units under the conditions of use of said emulsion; said emulsion comprising an organic phase dispersed in an aqueous phase and, optionally, comprising at least one outer surfactant or at least one outer amphiphilic polymer.

23. The process as claimed in claim 22, wherein the nonionic hydrophobic block of the block copolymer is obtained from at least one monomer selected from the group consisting of:
linear, branched, cyclic or aromatic monocarboxylic or polycarboxylic acid esters, comprising at least one ethylenic unsaturation;
saturated carboxylic acid esters comprising 8 to 30 carbon atoms, optionally bearing a hydroxyl group;
α,β-ethylenically unsaturated nitrites, vinyl ethers, vinyl esters, vinylaromatic monomers, vinyl halides or vinylidene halides;
linear or branched, aromatic or non-aromatic hydrocarbon-based monomers comprising at least one ethylenic unsaturation; and
propylene oxide or butylene oxide.

24. The process as claimed in claim 22, wherein the block containing cationic units of the block copolymer is obtained from at least one monomer selected from the group consisting of:
aminoalkyl (meth)acrylates and aminoalkyl(meth)acrylamides;
monomers comprising at least one secondary, tertiary or quaternary amine function, or a heterocyclic group containing a nitrogen atom, vinylamine or ethyleneimine; and
diallyldialkylammonium salts.

25. The process as claimed in claim 22, wherein either or both of the two blocks comprise one or more nonionic hydrophilic monomers being ethylene oxide; linear, branched, cyclic or aromatic monocarboxylic or polycarboxylic acid amides comprising at least one ethylenic unsaturation; hydrophilic esters derived from (meth)acrylic acid; vinyl esters for obtaining polyvinyl alcohol blocks after hydrolysis; or monomers of sugar type.

26. The process as claimed in claim 22, wherein the block copolymer is a linear copolymer comprising two blocks.

27. The process as claimed in claim 22, wherein the block copolymer is a comb or grafted copolymer comprising a nonionic skeleton, which is optionally hydrophilic, comprising cationic pendent segments and nonionic hydrophobic segments.

28. The process as claimed in claim 22, wherein the block copolymer is a copolymer of starburst structure.

29. The process as claimed in claim 22, wherein the block copolymer has a weight-average molar mass of between 2000 and 100 000 g/mol.

30. The process as claimed in claim 29, wherein the weight-average molar mass is between 2000 and 20 000 g/mol.

31. The process as claimed in claim 29, wherein the block copolymer is such that the blocks containing cationic units have a weight-average molar mass representing at least twice the mass of the nonionic hydrophobic blocks.

32. The process as claimed in claim 22, wherein the cationic block(s) of the block copolymer is (are) such that at least 5 mol % of the monomers from which the block(s) is (are) obtained are cationic monomers.

33. The process as claimed in claim 22, wherein the block(s) containing cationic units of the block copolymer is (are) obtained from cationic monomers and the cationic block(s) is (are) optionally homopolymers.

34. The process as claimed in claim 22, wherein the organic phase of the emulsion comprises an inner aqueous phase dispersed in said organic phase; the combination of the organic phase and of the inner aqueous phase comprising at least one inner surfactant and/or at least one inner amphiphilic polymer.

35. The process as claimed in claim 22, wherein the organic phase of the emulsion comprises an inner organic phase dispersed in said organic phase; the combination of the organic phase and of the inner organic phase comprising at least one inner stabilizing polymer.

36. The process as claimed in claim 22, wherein the block copolymer represents a content of from 0.1% to 30% by weight relative to the weight of the organic phase.

37. The process as claimed in claim 22, wherein the block copolymer is added during the preparation of the emulsion of the organic phase and of the aqueous phase; the organic phase optionally comprising a dispersed inner aqueous phase or a dispersed inner organic phase.

38. The process as claimed in claim 22, wherein the block copolymer is added once the emulsion of the organic phase and of the aqueous phase has been prepared; the organic phase optionally comprising a dispersed inner aqueous phase or a dispersed inner organic phase.

39. The process as claimed in claim 22, wherein the emulsion is a constituent component of a formulation for treating the skin or the hair, textiles, for the plant protection sector or the treatment of metal.

40. The process as claimed in claim 39, wherein:
the emulsion to be deposited represents a constituent component of an aqueous liquid detergent formulation for washing articles made of textile fiber, comprising at least one anionic surfactant,
the emulsion is a simple emulsion, the organic phase of which comprises at least one organic or organosilicon hydrophobic active material, or
the emulsion is intended to be deposited on the surface of articles made of textile fiber during a washing operation.

41. The process as claimed in claim 40, wherein the organic or organosilicon hydrophobic active material is oils, waxes of plant origin, fatty acid esters, mineral oils, paraffin waxes, nonionic silicone oils, nonionic silicone waxes, aminosilicone oils, or silicone oils containing hindered piperidyl function(s).

42. The process or the process as claimed in claim 41, wherein the organic or organosilicon hydrophobic active material in emulsion gives textile surfaces onto which it is deposited creaseproof properties, easy-ironing properties, antisoiling properties, or better abrasion resistance.
